(19) United States
(12) Patent Application Publication
(10) Pub. No.: US 2004/0029875 A1
(43) Pub. Date: Feb. 12, 2004

(54) Novel aminotriazolone compounds, method for preparing same and pharmaceutical compositions containing same

(76) Inventors: Jean-Luc Fauchere, Saint Cloud (FR); Jean-Claude Ortuno, Bois D'Arcy (FR); Nigel Levens, Vaucresson (FR); Susana Chamorro, Montesson (FR); Jean Albert Boutin, Suresnes (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/399,098

(22) PCT Filed: Oct. 11, 2001
(86) PCT No.: PCT/FR01/03133

(30) Foreign Application Priority Data
Oct. 13, 2000 (FR) 00/13125

(51) Int. Cl. A61K 31/541; A61K 31/5377; A61K 31/496; A61K 31/454; A61K 31/4196; C07D 417/02; C07D 413/02; C 07D 4 3/02

(52) U.S. Cl. 514/227.8; 514/236.2; 514/254.05; 514/326; 514/383; 544/60; 544/132; 544/366; 546/210; 548/263.2

(57) ABSTRACT

A compound of formula (I):

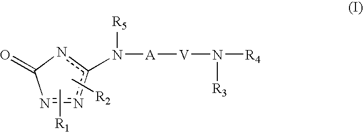

wherein:

R₁ and R₂ represent hydrogen or a group as defined in the description,

R₃ represents hydrogen or alkyl, alkenyl, alkynyl, aryl, heteroaryl, cycloalkyl or heterocycloalkyl,

R₄ represents a group of formula (II):

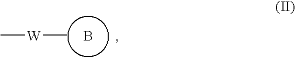

wherein W and B are as defined in the description,

R₅ represents hydrogen or alkyl,

A represents a group selected from -A₂-, -A₁-A₂-, -A₂-A₁- and -A₁-A₂-A₁-,

V is as defined in the description.

The present invention relates to new aminotriazolone compounds, to a process for their preparation and to pharmaceutical compositions containing them.

The compounds of the present invention have a novel structure and are used in the treatment of pathologies associated with neuropeptide Y (NPY).

Neuropeptide Y (NPY) is a peptide of 36 amino acids related to peptide YY (PYY) and to pancreatic polypeptides (PP). Originally isolated from pigs' brains (Proc. Natl. Acad. Sci., 1982, 79, 5485), NPY is widely distributed in mammals in the central and peripheral nervous systems. This neurotransmitter is present in high concentrations in the nerve fibres of the brain and also of the heart, the sympathetic ganglia, the blood vessels and the smooth muscles of the vas deferens and of the gastrointestinal tract. It is responsible for various physiological effects that are exerted by means of specific (Y) receptors. The latter form a heterogeneous group, 6 sub-types of which have been identified to date: Y₁ to Y₆ (Pharmacological Reviews, 1998, 50, 143). NPY is involved in eating behaviour, strongly stimulating food intake (Proc. Natl. Acad. Sci., 1985, 82, 3940) or exerting a regulatory role on the HPA (hypothalamic-pituitary-adrenal) axis (J. of Neuroendocrinol., 1995, 7, 273). It also exhibits anxiolytic and sedative properties (Neuropsychopharmacology, 1993, 8, 357) and a strong vasoconstrictive ability (Eur. J. Pharmacol., 1984, 85 519) which induces an increase in blood pressure, and it also has an effect on circadian rhythm (Neuroscience and Biobehavioral Reviews, 1995, 19, 349).

Various NPY receptor ligands have been described recently. By way of example, there may be mentioned cyclic peptide compounds (WO 9400486), amino acid compounds of arginine (WO 9417035) and non-peptide compounds (WO 9827063).

In addition to the fact the compounds of the invention are new, they have demonstrated an in vivo inhibitory action on food intake and weight gain. That effect is exerted by means of binding to the NPY receptors. It will thus be possible to use the compounds of the invention in treatment of pathologies that requires a ligand of receptors of NPY, especially in the treatment of pathologies associated with eating behaviour disorders or energy balance disorders, such as diabetes, obesity, bulimia and anorexia nervosa, and also in the treatment of arterial hypertension, anxiety, depression, epilepsy, sexual dysfunctions and sleep disorders.

More specifically, the present invention relates to compounds of formula (I):

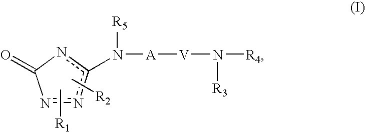

wherein:

R₁ and R₂ each independently of the other represents a hydrogen atom or an unsubstituted or substituted alkyl group, an unsubstituted or substituted alkenyl group, an unsubstituted or substituted alkynyl group, an unsubstituted or substituted aryl group, an unsubstituted or substituted heteroaryl group, an unsubstituted or substituted cycloalkyl group, or an unsubstituted or substituted heterocycloalkyl group, it being understood that at least one of groups R₁ and R₂ is other than a hydrogen atom,

R₃ represents a hydrogen atom or an unsubstituted or substituted alkyl group, an unsubstituted or substituted alkenyl group, an unsubstituted or substituted alkynyl group, an unsubstituted or substituted aryl group, an unsubstituted or substituted heteroaryl group, an unsubstituted or substituted cycloalkyl group, or an unsubstituted or substituted heterocycloalkyl group,

R₄ represents a group of formula (II):

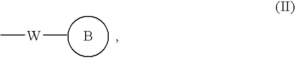

wherein W represents a bond or an alkylene chain containing from 1 to 6 carbon atoms and B represents a mono- or poly-cyclic, aromatic or non-aromatic, group containing from 3 to 10 ring atoms, which may include from 1 to 3 hetero atoms selected from oxygen, sulphur and nitrogen, and containing at least one oxo, —COR (wherein R represents a hydrogen atom or an alkyl, alkoxy, amino, alkylamino or dialkylamino group) or hydroxy substituent, and which may contain one or more unsaturations and/or one or more substituents (in addition to the oxo, COR or hydroxy group defined above) selected from alkyl, alkoxy, aryl, arylalkyl and halogen atoms,

R₅ represents a hydrogen atom or an alkyl group,

A represents a group selected from -A₂-, -A₁-A₂-, -A₂-A₁- and -A₁-A₂-A₁-, wherein A₁ is an alkylene, alkenylene or alkynylene group and A₂ represents an unsubstituted or substituted phenylene group, an unsubstituted or substituted naphthylene group, an unsubstituted or substituted cycloalkylene group, an unsubstituted or substituted heteroarylene group, or an unsubstituted or substituted heterocycloalkylene group,

V represents a bond or a group —CH₂—, —CO—, —CS—, —CH₂—NH— or —CH═N—, or V and R₃, together with the groups -A- and —N—R₄ carrying them, form a group -A-CH═N—R₄,

it being understood that,

the term “alkyl” denotes a linear or branched group having from 1 to 6 carbon atoms,

the term “alkylene” denotes a linear or branched bivalent radical containing from 1 to 6 carbon atoms,

the term “alkenyl” denotes a linear or branched group containing from 2 to 6 carbon atoms and from 1 to 3 double bonds,

the term “alkenylene” denotes a linear or branched bivalent radical containing from 2 to 6 carbon atoms and from 1 to 3 double bonds,

the term “alkynyl” denotes a linear or branched group containing from 2 to 6 carbon atoms and from 1 to 3 triple bonds,

the term “alkynylene” denotes a linear or branched bivalent radical containing from 2 to 6 carbon atoms and from 1 to 3 triple bonds,

the term “aryl” denotes a phenyl, naphthyl, biphenyl, dihydronaphthyl or tetrahydronaphthyl group,

the term “heteroaryl” denotes an unsaturated or partially unsaturated mono- or bi-cyclic group having from 5 to 11 ring members, containing from 1 to 4 hetero atoms selected from nitrogen, oxygen and sulphur,

the terms “phenylene” and “naphthylene” denote bivalent phenyl and naphthyl radicals, respectively,

the term “heteroarylene” denotes a bivalent heteroaryl radical, heteroaryl being as defined hereinbefore,

the term “heterocycloalkyl” denotes a saturated mono- or bi-cyclic group having from 4 to 11 ring members, containing from 1 to 4 hetero atoms selected from nitrogen, oxygen and sulphur,

the term “heterocycloalkylene” denotes a saturated mono- or bi-cyclic bivalent radical having from 4 to 11 ring members, containing from 1 to 4 hetero atoms selected from nitrogen, oxygen and sulphur,

the term “cycloalkyl” denotes a saturated cyclic group containing from 3 to 8 carbon atoms,

the term “cycloalkylene” denotes a saturated bivalent cyclic group containing from 3 to 8 carbon atoms,

the expression “substituted” applied to the terms “aryl” or “heteroaryl” means that those groups are substituted on their cyclic moiety by from 1 to 5 identical or different substituents selected from linear or branched (C₁-C₆)alkyl, linear or branched (C₁-C₆)alkoxy, halogen, hydroxy, linear or branched (C₁-C₆)-perhaloalkyl, nitro, amino (unsubstituted or substituted by one or two groups selected from linear or branched (C₁-C₆)alkyl, aryl and heteroaryl), linear or branched (C₁-C₆)acyl, aminocarbonyl (optionally substituted on the nitrogen atom by one or two linear or branched (C₁-C₆)alkyl groups), linear or branched (C₁-C₆)acylamino, linear or branched (C₁-C₆)alkoxy-carbonyl, formyl, carboxy, sulpho, sulphino, sulphamoyl, nitrile, linear or branched (C₁-C₆)-aminoalkyl (optionally substituted on the nitrogen atom by one or two linear or branched (C₁-C₆)alkyl groups), linear or branched (C₁-C₆)-thioalkyl (optionally substituted on the sulphur atom by a linear or branched (C₁-C₆)alkyl group) and hydroxyalkyl (optionally substituted on the oxygen atom by a linear or branched (C₁-C₆)alkyl group),

the expression “substituted” applied to the terms “alkyl”, “alkenyl” or “alkynyl” means that those groups may be substituted by one or more groups selected from hydroxy, unsubstituted or substituted cycloalkyl, unsubstituted or substituted aryl, unsubstituted or substituted heteroaryl, unsubstituted or substituted heterocycloalkyl and halogen atoms,

the expression “substituted” applied to the terms “phenylene”, “naphthylene” or “heteroarylene” means that those groups are substituted by from one to three identical or different groups selected from linear or branched (C₁-C₆)alkyl, linear or branched (C₁-C₆)alkoxy, halogen, hydroxy, linear or branched (C₁-C₆)-perhaloalkyl, nitro, amino (unsubstituted or substituted by one or two groups selected from linear or branched (C₁-C₆)alkyl, aryl and heteroaryl), linear or branched (C₁-C₆)acyl, formyl, carboxy, linear or branched (C₁-C₆)alkoxy-carbonyl, aminocarbonyl (optionally substituted on the nitrogen atom by one or two linear or branched (C₁-C₆)alkyl groups), linear or branched (C₁-C₆)acylamino and nitrile,

their enantiomers, diastereoisomers, and addition salts thereof with a pharmaceutically acceptable acid or base.

More especially, the invention relates to compounds of formula (I) wherein A represents a phenylene group and, more especially, an unsubstituted phenylene group.

Preferred groups R₁ and R₂ are a hydrogen atom and an aryl group such as, for example, pyridyl or phenyl, those groups being unsubstituted or substituted.

The invention relates more especially to compounds of formula (I) wherein R₃ and R₅ represent a hydrogen atom.

Preferred groups R₄ are groups of formula (II′):

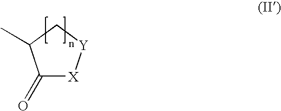

wherein n is 0, 1, 2 or 3, and X represents an oxygen or sulphur atom and in this case Y represents a group CH₂, or X represents a group NH and in this case Y represents a group CH₂ or an oxygen atom.

Other preferred groups R₄ are groups of formula (II″):

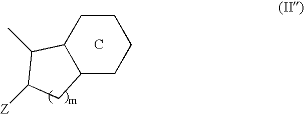

wherein m is 0, 1 or 2, and Z represents a hydroxy or amino group and C represents an optionally substituted, aromatic 6-membered ring which may contain from 1 to 3 nitrogen atoms.

Advantageously, the invention relates to compounds of formula (I) wherein V represents a group CO or CH₂.

More especially, the invention relates to compounds of formula (I) that are:

N-[(3R)-2-oxotetrahydro-3-furanyl]-4-({5-oxo-1-[3-(trifluoromethyl)phenyl]-4,5-dihydro-1H-1,2,4-triazol-3-yl}amino)benzamide

4-{[1-(3-methylphenyl)-5-oxo-4,5-dihydro-1H-1,2,4-triazol-3-yl]amino}-N-[(3R)-2-oxotetrahydro-3-furanyl]benzamide

4-{[1-(3-methylphenyl)-5-oxo-4,5-dihydro-1H-1,2,4-triazol-3-yl]amino}-N-[(3R)-2-oxotetrahydro-3-thienyl]benzamide

4-{([1-(3-chlorophenyl)-5-oxo-4,5-dihydro-1H-1,2,4-triazol-3-yl]amino}-N-[(3R)-2-oxotetrahydro-3-thienyl]benzamide trifluoroacetate

4-{[1-(3-chlorophenyl)-5-oxo-4,5-dihydro-1H-1,2,4-triazol-3-yl]amino}-N-[(3R)-2-oxotetrahydro-3-furanyl]benzamide

4-{[1-(4-chlorophenyl)-5-oxo-4,5-dihydro-1H-1,2,4-triazol-3-yl]amino}-N-[(3R)-2-oxotetrahydro-3-furanyl]benzamide

3-[(4-{[1-(3-methylphenyl)-5-oxo-4,5-dihydro-1H-1,2,4-triazol-3-yl]amino}benzyl)amino]-2-azepanone trifluoroacetate.

Other preferred compounds are the following compounds of formula (I):

N-[(1R,2S)-2-hydroxy-2,3-dihydro-1H-inden-1-yl]-4-[(5-oxo-1-phenyl-4,5-dihydro-1H-1,2,4-triazol-3-yl)amino]benzamide

N-[(1R,2S)-2-hydroxy-2,3-dihydro-1H-inden-1-yl]-4-{[1-(3-methylphenyl)-5-oxo-4,5-dihydro-1H-1,2,4-triazol-3-yl]amino}benzamide

4-1{[1-(3-chlorophenyl)-5-oxo-4,5-dihydro-1H-1,2,4-triazol-3-yl]amino}-N-[(1R,2S)-1-hydroxy-2,3-dihydro-1H-inden-2-yl]benzamide

2-(3-chlorophenyl)-5-[4-({[(1S,2R)-2-hydroxy-2,3-dihydro-1H-inden-1-yl]amino}-methyl)anilino]-2,4-dihydro-3H-1,2,4-triazol-3-one trifluoroacetate

2-(3-chlorophenyl)-5-[4-({[(1R,2S)-2-hydroxy-2,3-dihydro-1H-inden-1-yl]amino}-methyl)anilino]-2,4-dihydro-3H-1,2,4-triazol-3-one trifluoroacetate.

The enantiomers and diastereoisomers and addition salts with a pharmaceutically acceptable acid or base of the preferred compounds of the invention form an integral part of the invention.

The present invention relates also to a process for the preparation of compounds of formula (I), which process is characterised in that there is used as starting material the compound of formula (III):

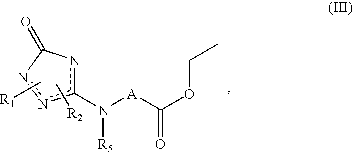

wherein R₁, R₂, R₅ and A are as defined hereinbefore,

which is hydrolysed in a basic medium to yield the compound of formula (IV):

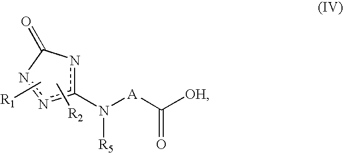

wherein R₁, R₂, R₅ and A are as defined hereinbefore,

with which there is condensed, in the presence of a coupling agent, an amine of formula NHR′₃R₄ (wherein R₄ is as defined hereinbefore and R′₃ may have any of the meanings of R₃ but cannot form a further bond together with V) to yield the compound of formula (I/a), a particular case of the compounds of formula (I):

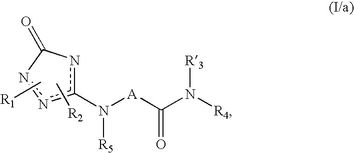

wherein R₁, R₂, R′₃, R₄, R₅ and A are as defined hereinbefore,

which is subjected to a thionating agent such as Lawesson's reagent to yield the compound of formula (I/b), a particular case of the compounds of formula (I):

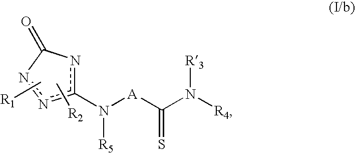

wherein R₁, R₂, R′₃, R₄, R₅ and A are as defined hereinbefore,

or which is subjected to a reducing agent to yield the compound of formula (I/c), a particular case of the compounds of formula (I):

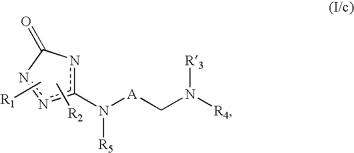

wherein R₁, R₂, R′₃, R₄, R₅ and A are as defined hereinbefore,

or with which compound of formula (IV) there is condensed, in the presence of a coupling agent, N,O-dimethylhydroxylamine, and which is then reduced in the presence of a reducing agent to yield the compound of formula (V):

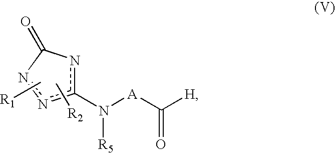

wherein R₁, R₂, R₅ and A are as defined hereinbefore,

with which there is condensed a compound of formula R₄NH₂, wherein R₄ is as defined hereinbefore, to yield the compound of formula (I/d), a particular case of the compounds of formula (I):

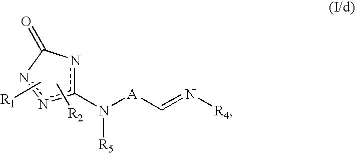

wherein R₁, R₂, R₄, R₅ and A are as defined hereinbefore,

which may be reduced to obtain the compound of formula (I/c′), a particular case of the compounds of formula (I/c):

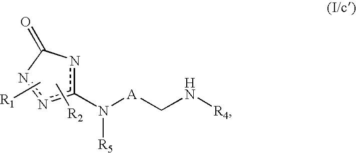

wherein R₁, R₂, R₄, R₅ and A are as defined hereinbefore,

or with which there is condensed a hydrazine of formula H₂N—NR′₃R₄ wherein R′₃ and R₄ are as defined hereinbefore,

under non-reductive conditions to yield the compound of formula (I/e), a particular case of the compounds of formula (I):

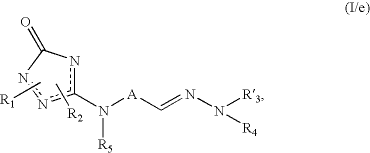

wherein R₁, R₂, R′₃, R₄, R₅ and A are as defined hereinbefore,

or in the presence of a reducing agent to yield the compound of formula (I/f), a particular case of the compounds of formula (I):

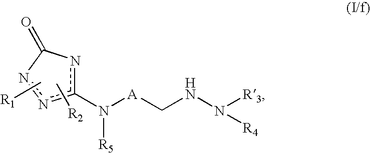

wherein R₁, R₂, R′₃, R₄, R₅ and A are as defined hereinbefore,

the totality of compounds (I/a) to (I/f) constituting the compound of formula (I), which may be purified, if desired, according to a conventional purification technique, which may be separated, where applicable, into its isomers (enantiomers and/or diastereoisomers) according to a conventional separation technique, and which is converted, where appropriate, into addition salts thereof with a pharmaceutically acceptable acid or base.

The compounds of formula (III) are readily accessible to the person skilled in the art by means of conventional chemical reactions or by methods described in the literature.

In particular, compounds of formula (III) may be obtained starting from the compound of formula (VI):

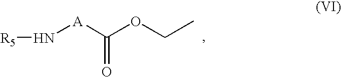

wherein A is as defined hereinbefore,

with which there is condensed, in a basic medium, an isothiocyanate of formula (VII):

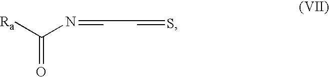

wherein Rₐ represents a linear or branched (C₁-C₆)alkoxy group,

to obtain the compound of formula (VIII):

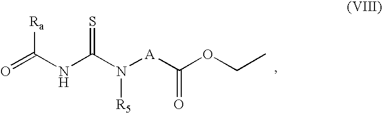

wherein A and Rₐ are as defined hereinbefore,

with which there is condensed, in the presence of a coupling agent and triphenylphosphine, an alcohol of formula R_{b}OH (wherein R_{b} represents a group R₁ or R₂ except for a hydrogen atom) to obtain the compound of formula (IX):

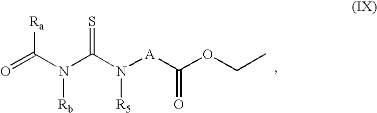

wherein A, Rₐ and R_{b} are as defined hereinbefore,

the totality of compounds (VIII) and (IX) being subjected to the action of a hydrazine of formula R₁NH—NHR₂ (wherein R₁ and R₂ are as defined hereinbefore) in the presence of a coupling agent to yield, after spontaneous cyclisation or cyclisation in an acid medium, the compound of formula (III).

The present invention relates also to pharmaceutical compositions comprising as active ingredient at least one compound of formula (I), alone or in combination with one or more inert, non-toxic, pharmaceutically acceptable excipients or carriers.

Among the pharmaceutical compositions according to the invention there may be mentioned more especially those that are suitable for oral, parenteral, nasal or transdermal administration, tablets or dragees, sublingual tablets, gelatin capsules, lozenges, suppositories, creams, ointments, dermal gels etc.

The useful dosage varies according to the age and weight of the patient, the nature and severity of the disorder and the administration route, which may be oral, nasal, rectal or parenteral. In general, the unit dose ranges from 0.05 to 500 mg per 24 hours for a treatment of from 1 to 3 administrations.

The following Examples illustrate the invention but do not limit it in any way.

The structures of the compounds described have been confirmed by conventional spectroscopic and spectrometric techniques.

A compound (1S*,2R*), for example, is understood to be a racemic mixture of 2 diastereoisomers having the absolute configurations (1S,2R) and (1R,2S).

A compound (1S*,2S*), for example, is understood to be a racemic mixture of 2 diastereoisomers having the absolute configurations (1S,2S) and (1R,2R).

### EXAMPLE 1

### 4-[(1-Cyclohexyl-5-oxo-4,5-dihydro-1H-1,2,4-triazol-3-yl)amino]-N-[(3S)-2-oxotetrahydro-3-furanyl]benzamide Trifluoroacetate

The procedure is as in Example 43, replacing the 3-(trifluoromethyl)phenylhydrazine in Step B by cyclohexylhydrazine.

Mass spectrum: ESI-MS:MH⁺=386

### EXAMPLE 2

### 4-[(1-Cyclohexyl-5-oxo-4,5-dihydro-1H-1,2,4-triazol-3-yl)amino]-N-({[(1S*,2R *)-2-hydroxycyclohexyl]methyl}benzamide

The procedure is as in Example 1, replacing the R(+)-α-amino-butyrolactone in Step D by (1R*,2S*)-2-(aminomethyl)cyclohexanol.

Mass spectrum: ESI-MS:MH⁺=414

### EXAMPLE 3

### 4-[(1-Cyclohexyl-5-oxo-4,5-dihydro-1H-1,2,4-triazol-3-yl)amino]-N-{[(1R*,2R *)-2-hydroxycyclohexyl]methyl}benzamide

The procedure is as in Example 1, replacing the R(+)-α-amino-butyrolactone in Step D by (1R*,2R*)-2-(aminomethyl)cyclohexanol.

Mass spectrum: ESI-MS:MH⁺=414

### EXAMPLE 4

### 4-[(5-Oxo-1-phenyl-4,5-dihydro-1H-1,2,4-triazol-3-yl)amino]-N-[(3R)-2-oxotetrahydro-3-furanyl]benzamide

The procedure is as in Example 43, replacing the 3-(trifluoromethyl)phenylhydrazine in Step B by phenylhydrazine.

Mass spectrum : ESI-MS:MH⁺=378

### EXAMPLE 5

### Ethyl (1R*,2S*)-2-({4-[(5-oxo-1-phenyl-4,5-dihydro-1H-1,2,4-triazol-3-yl)amino]benzoyl}amino)cyclohexanecarboxylate

The procedure is as in Example 4, replacing the R(+)-α-amino-butyrolactone in Step D by ethyl (1R*,2S*)-2-aminocyclohexanecarboxylate.

Mass spectrum: ESI-MS:MH⁺=473

### EXAMPLE 6

### Ethyl (1R*,2R*)-2-({4-[(5-oxo-1-phenyl-4,5-dihydro-1H-1,2,4-triazol-3-yl)amino]benzoyl}amino)cyclohexanecarboxylate

The procedure is as in Example 4, replacing the R(+)-α-amino-butyrolactone in Step D by ethyl (1R*,2R*)-2-aminocyclohexanecarboxylate.

Mass spectrum: ESI-MS:MH⁺=450

### EXAMPLE 7

### N-(2-oxo-3-azepanyl)-4-[(5-oxo-1-phenyl-4,5-dihydro-1H-1,2,4-triazol-3-yl)amino]benzamide

The procedure is as in Example 4, replacing the R(+)-α-amino-butyrolactone in Step D by 3-amino-2-azepanone.

Mass spectrum: ESI-MS:MH⁺=407

### EXAMPLE 8

### N-[(1R,2S)-2-hydroxy-2,3-dihydro-1H-inden-1-yl]-4-[(5-oxo-1-phenyl-4,5-dihydro-1H-1,2,4-triazol-3-yl)amino]benzamide

The procedure is as in Example 4, replacing the R(+)-α-amino-butyrolactone in Step D by (1R,2S)-1-amino-2,3-dihydro-1H-inden-2-ol.

Mass spectrum: ESI-MS:MH⁺=428

### EXAMPLE 9

### N-[(1R,2S)-2-hydroxy-2,3-dihydro-1H-inden-1-yl]-3-[(5-oxo-1-phenyl-4,5-dihydro-1H-1,2,4-triazol-3-yl)amino]benzamide

The procedure is as in Example 43, replacing the ethyl 4-aminobenzoate in Step A by ethyl 3-aminobenzoate, replacing the 3-(trifluoromethyl)phenylhydrazine in Step B by phenylhydrazine, and replacing the R(+)-α-amino-butyrolactone in Step D by (1R,2S)-1-amino-2,3-dihydro-1H-inden-2-ol.

Mass spectrum: ESI-MS:MH⁺=428

### EXAMPLE 10

### N-[(1S,2R)-2-hydroxy-2,3-dihydro-1H-inden-1-yl]-3-[(5-oxo-1-phenyl-4,5-dihydro-1H-1,2,4-triazol-3-yl)amino]benzamide

The procedure is as in Example 9, replacing the (1R,2S)-1-amino-2,3-dihydro-1H-inden-2-ol in Step D by (1S,2R)-1-amino-2,3-dihydro-1H-inden-2-ol.

### EXAMPLE 11

### 4-{[5-Oxo-1-(2-pyridyl)-4,5-dihydro-1H-1,2,4-triazol-3-yl]amino}-N-[(3R)-2-oxotetrahydro-3-furanyl]benzamide hydrochloride

The procedure is as in Example 43, replacing the 3-(trifluoromethyl)phenylhydrazine in Step B by 2-hydrazinopyridine.

Mass spectrum: ESI-MS:MH⁺=381

### EXAMPLE 12

### 4-{[5-Oxo-1-(4-pyridyl)-4,5-dihydro-1H-1,2,4-triazol-3-yl]amino}-N-[(3R)-2-oxotetrahydro-3-furanyl]benzamide Hydrochloride

The procedure is as in Example 11, replacing the 2-hydrazinopyridine in Step B by 4-hydrazinopyridine.

### EXAMPLE 13

### 4-{[5-Oxo-1-(2-pyridyl)-4,5-dihydro-1H-1,2,4-triazol-3-yl]amino}-N-[(3R)-2-oxotetrahydro-3-thienyl]benzamide Trifluoroacetate

The procedure is as in Example 11, replacing the R(+)-α-amino-butyrolactone in Step D by (3R)-3-aminodihydro-2(3H)-thiophenone.

Mass spectrum: ESI-MS:MH⁺=397

### EXAMPLE 14

### N-[1(1S*,2S*)-2-hydroxycyclobexyl]-4-{[5-oxo-1-(2-pyridyl)-4,5-dihydro-1H-1,2,4-triazol-3-yl]amino}benzamide trifluoroacetate

The procedure is as in Example 13, replacing the (3R)-3-aminodihydro-2(3H)-thiophenone in Step D by (1S*,2S*)-2-aminocyclohexanol.

Mass spectrum: ESI-MS:MH⁺=395

### EXAMPLE 15

### N-{[(1R*,2R*)-2-hydroxycyclohexyl]methyl}-4-{[5-oxo-1-(2-pyridyl)-4,5-dihydro-1H-1,2,4-triazol-3-yl]amino}benzamide Trifluoroacetate

The procedure is as in Example 13, replacing the (3R)-3-aminodihydro-2(3H)-thiophenone in Step D by (1R*,2R*)-2-(aminomethyl)cyclohexanol.

Mass spectrum: ESI-MS:MH⁺=409

### EXAMPLE 16

### N-{[(1S*,2R*)-2-hydroxycyclohexyl]methyl}-4-{[5-oxo-1-(2-pyridyl)-4,5-dihydro-1H-1,2,4-triazol-3-yl]amino}benzamide

The procedure is as in Example 13, replacing the (3R)-3-aminodihydro-2(3H)-thiophenone in Step D by (1S*,2R*)-2-(aminomethyl)cyclohexanol.

### EXAMPLE 17

### N-(2-oxo-3-azepanyl)-4-{[5-oxo-1-(2-pyridyl)-4,5-dihydro-1H-1,2,4-triazol-3-yl]amino}benzamide Trifluoroacetate

The procedure is as in Example 13, replacing the (3R)-3-aminodihydro-2(3H)-thiophenone in Step D by 3-amino-2-azepanone.

Mass spectrum: ESI-MS:MH⁺=408

### EXAMPLE 18

### 4-{[1-(3-Methylphenyl)-5-oxo-4,5-dihydro-1H-1,2,4-triazol-3-yl]amino}-N-[(3R)-2-oxotetrahydro-3-furanyl]benzamide

The procedure is as in Example 43, replacing the 3-(trifluoromethyl)phenylhydrazine in Step B by 1-(3-methylphenyl)hydrazine.

Mass spectrum: ESI-MS:MH⁺=394

### EXAMPLE 19

### 4-{[1-(3-Methylphenyl)-5-oxo-4,5-dihydro-1H-1,2,4-triazol-3-yl]amino}-N-[(3R)-2-oxotetrahydro-3-thienyl]benzamide

The procedure is as in Example 18, replacing the R(+)-α-amino-butyrolactone in Step D by (3R)-3-aminodihydro-2(3H)-thiophenone.

Mass spectrum: ESI-MS:MH⁺=410

### EXAMPLE 20

### N-[(1S*,2S*)-2-hydroxycyclohexyl]-4-{[1-(3-methylphenyl)-5-oxo-4,5-dihydro-1H-1,2,4-triazol-3-yl]amino}benzamide Trifluoroacetate

The procedure is as in Example 18, replacing the R(+)-α-amino-butyrolactone in Step D by (1S*,2S*)-2-aminocyclohexanol.

Mass spectrum: ESI-MS:MH⁺=408

### EXAMPLE 21

### 4-{1[1-(3-Methylphenyl)-5-oxo-4,5-dihydro-1H-1,2,4-triazol-3-yl]amino}-N-(2-oxo-3-azepanyl)benzamide

The procedure is as in Example 18, replacing the R(+)-α-amino-butyrolactone in Step D by 3-amino-2-azepanone.

Mass spectrum: ESI-MS:MH⁺=421

### EXAMPLE 22

### N-[(1R,2S)-2-hydroxy-2,3-dihydro-1H-inden-1-yl]-4-{[1-(3-methylphenyl)-5-oxo-4,5-dihydro-1H-1,2,4-triazol-3-yl]amino}benzamide

The procedure is as in Example 18, replacing the R(+)-α-amino-butyrolactone in Step D by (1R,2S)-1-amino-2,3-dihydro-1H-inden-2-ol.

Mass spectrum: ESI-MS:MH⁺=442

### EXAMPLE 23

### 4-{[1-(3,5-Dimethylphenyl)-5-xo-4,5-dihydro-1H-1,2,4-triazol-3-yl]-amino}-N-[(1R,2S)-2-hydroxy-2,3-dihydro-1H-inden-1-yl]benzamide

The procedure is as in Example 22, replacing the 1-(3-methylphenyl)hydrazine in Step B by 1-(3,5-dimethylphenyl)hydrazine.

Mass spectrum: ESI-MS:MH⁺=455

### EXAMPLE 24

### N-[(1R,2S)-2-hydroxy-2,3-dihydro-1H-inden-1-yl]-4-{[1-(4-isopropyl-phenyl)-5-oxo-4,5-dihydro-1H-1,2,4-triazol-3-yl]amino}benzamide

The procedure is as in Example 22, replacing the 1-(3-methylphenyl)hydrazine in Step B by 1-(4-isopropylphenyl)hydrazine.

Mass spectrum: ESI-MS:MH⁺=470

### EXAMPLE 25

### 4-{[1-(4-Methoxyphenyl)-5-oxo-4,5-dihydro-1H-1,2,4-triazol-3-yl]-amino}-N-[(3R)-2-oxotetrahydro-3-furanyl]benzamide

The procedure is as in Example 43, replacing the 3-(trifluoromethyl)phenylhydrazine in Step B by 1-(4-methoxyphenyl)hydrazine.

Mass spectrum: ESI-MS:MH⁺=410

### EXAMPLE 26

### 4-{1[1-(4-Methoxyphenyl)-5-oxo-4,5-dihydro-1H-1,2,4-triazol-3-yl]-amino}-N-(2-oxo-3-azepanyl)benzamide

The procedure is as in Example 25, replacing the R(+)-α-amino-butyrolactone in Step D by 3-amino-2-azepanone.

Mass spectrum: ESI-MS:MH⁺=437

### EXAMPLE 27

### N-[(1R,2S)-1-hydroxy-2,3-dihydro-1H-inden-2-yl]-4-([1-(3-methoxyphenyl)-5-oxo-4,5-dihydro-1H-1,2,4-triazol-3-yl]amino)benzamide

The procedure is as in Example 43, replacing the 3-(trifluoromethyl)phenylhydrazine in Step B by 1-(3-methoxyphenyl)hydrazine, and replacing the R(+)-α-amino-butyrolactone in Step D by (1R,2S)-1-amino-2,3-dihydro-1H-inden-2-ol.

Mass spectrum: ESI-MS:M-H⁻=456

### EXAMPLE 28

### 4-{[1-(4-Fluorophenyl)-5-oxo-4,5-dihydro-1H-1,2,4-triazol-3-yl]amino)-N-[(1R,2S)-2-hydroxy-2,3-dihydro-1H-inden-1-yl]benzamide

The procedure is as in Example 43, replacing the 3-(trifluoromethyl)phenylhydrazine in Step B by 1-(4-fluorophenyl)hydrazine, and replacing the R(+)-α-amino-butyrolactone in Step D by (I R,2S)-1-amino-2,3-dihydro-1H-inden-2-ol.

Mass spectrum: ESI-MS:MH⁺=446

### EXAMPLE 29

### 4-{[1-(3-Chlorophenyl)-5-oxo-4,5-dihydro-1H-1,2,4-triazol-3-yl]amino)-N-[(3R)-2-oxotetrahydro-3-thienyl]benzamide trifluoroacetate

The procedure is as in Example 43, replacing the 3-(trifluoromethyl)phenylhydrazine in Step B by 1-(3-chlorophenyl)hydrazine, and replacing the R(+)-α-aminobutyrolactone in Step D by (3R)-3-aminodihydro-2(3H)-thiophenone.

Mass spectrum: ESI-MS:M-H=428

### EXAMPLE 30

### 4-{[1-(3-Chlorophenyl)-5-oxo-4,5-dihydro-1H-1,2,4-triazol-3-yl]amino)-N-[(3R)-2-oxotetrahydro-3-furanyl]benzamide

The procedure is as in Example 29, replacing the (3R)-3-aminodihydro-2(3H)-thiophenone in Step D by R(+)-α-aminobutyrolactone.

Mass spectrum: ESI-MS:MH⁺=414

### EXAMPLE 31

### N-[(1S*,2R*)-2-(aminocarbonyl)cyclopentyl]-4-{[1-(3-chlorophenyl)-5-oxo-4,5-dihydro-1H-1,2,4-triazol-3-yl]amino}benzamide

The procedure is as in Example 29, replacing the (3R)-3-aminodihydro-2(3H)-thiophenone in Step D by (1S*,2R*)-2-aminocyclopentanecarboxamide.

Mass spectrum: ESI-MS:M-H=439

### EXAMPLE 32

### 4-{[1-(3-Chlorophenyl)-5-oxo-4,5-dihydro-1H-1,2,4-triazol-3-yl]amino}-N-(2-oxo-3-azepanyl)benzamide

The procedure is as in Example 29, replacing the (3R)-3-aminodihydro-2(3H)-thiophenone in Step D by 3-amino-2-azepanone.

Mass spectrum: ESI-MS:MH+F=441

### EXAMPLE 33

### 4-{1[1-(3-Chlorophenyl)-5-oxo-4,5-dihydro-1H-1,2,4-triazol-3-yl]amino}-N-[(1R,2S)-1-hydroxy-2,3-dihydro-1H-inden-2-yl]benzamide

The procedure is as in Example 29, replacing the (3R)-3-aminodihydro-2(3H)-thiophenone in Step D by (1R,2S)-1-amino-2,3-dihydro-1H-inden-2-ol.

Mass spectrum: ESI-MS:MH⁺=460

### EXAMPLE 34

### 4-{[1-(4-Chlorophenyl)-5-oxo-4,5-dihydro-1H-1,2,4-triazol-3-yl]amino}-N-[(3R)-2-oxotetrahydro-3-furanyl]benzamide

The procedure is as in Example 43, replacing the 3-(trifluoromethyl)phenylhydrazine in Step B by 1-(4-chlorophenyl)hydrazine.

Mass spectrum: ESI-MS:MH⁺=414

### EXAMPLE 35

### 4-{[1-(4-Chlorophenyl)-5-oxo-4,5-dihydro-1H-1,2,4-triazol-3-yl]amino)-N-{[(1S*,2R *)-2-hydroxycyclohexyl]methyl}benzamide

The procedure is as in Example 34, replacing the R(+)-α-amino-butyrolactone in Step D by (1I*,2R *)-2-(aminomethyl)cyclohexanol.

Mass spectrum: ESI-MS:MH⁺=442

### EXAMPLE 36

### 4-{[1-(4-Chlorophenyl)-5-oxo-4,5-dihydro-1H-1,2,4-triazol-3-yl]amino}-N-{[(1R*,2R *)-2-hydroxycyclohexyl]methyl}benzamide

The procedure is as in Example 34, replacing the R(+)-α-amino-butyrolactone in Step D by (1R*,2R *)-2-(aminomethyl)cyclohexanol.

Mass spectrum: ESI-MS:MH⁺=442

### EXAMPLE 37

### Ethyl (1S*,2S*)-2-[(4-{[1-(4-chlorophenyl)-5-oxo-4,5-dihydro-1H-1,2,4-triazol-3-yl]amino}benzoyl)amino]cyclohexanecarboxylate

The procedure is as in Example 34, replacing the R(+)-α-amino-butyrolactone in Step D by ethyl (1S*,2S*)-2-aminocyclohexanecarboxylate.

Mass spectrum: ESI-MS:M-H⁻=482

### EXAMPLE 38

### Ethyl (1R*,2S*)-2-[(4-{1-(4-chlorophenyl)-5-oxo-4,5-dihydro-1H-1,2,4-triazol-3-yl]amino}benzoyl)aminolcyclohexanecarboxylate

The procedure is as in Example 34, replacing the R(+)-α-amino-butyrolactone in Step D is by ethyl (1R*,2S*)-2-aminocyclohexanecarboxylate.

### EXAMPLE 39

### 4-({[1-(3-Chlorophenyl)-5-oxo-4,5-dihydro-1H-1,2,4-triazol-3-yl]-amino}methyl)-N-[(1R,2S)-2-hydroxy-2,3-dihydro-1H-inden-1-yl]-benzamide

The procedure is as in Example 43, replacing the ethyl 4-aminobenzoate in Step A by ethyl 4-(aminomethyl)benzoate, replacing the 3-(trifluoromethyl)phenylhydrazine in Step B by 1-(3-chlorophenyl)hydrazine, and replacing the R(+)-α-amino-butyrolactone in Step D by (1R,2S)-1-amino-2,3-dihydro-1H-inden-2-ol.

Mass spectrum: ESI-MS:MH⁺=476

### EXAMPLE 40

### 4-({[11-(3-Chlorophenyl)-5-oxo-4,5-dihydro-1H-1,2,4-triazol-3-yl]-amino}methyl)-N-[(1S,2R)-2-hydroxy-2,3-dihydro-1H-inden-1-yl]-benzamide

The procedure is as in Example 39, replacing the (1R,2S)-1-amino-2,3-dihydro-1H-inden-2-ol in Step D by (1S,2R)-1-amino-2,3-dihydro-1H-inden-2-ol.

Mass spectrum: ESI-MS:MH⁺=476

### EXAMPLE 41

### 4-{[1-(3,5-Dichlorophenyl)-5-oxo-4,5-dihydro-1H-1,2,4-triazol-3-yl]-amino}-N-[(1R,2S)-2-hydroxy-2,3-dihydro-1H-inden-1-yl]benzamide

The procedure is as in Example 43, replacing the 3-(trifluoromethyl)phenylhydrazine in Step B by 1-(3,5-dichlorophenyl)hydrazine, and replacing the R(+)-α-amino-butyrolactone in Step D by (1R,2S)-1-amino-2,3-dihydro-1H-inden-2-ol.

### EXAMPLE 42

### N-(2-oxo-3-azetidinyl)-4-({5-oxo-1-[3-(trifluoromethyl)phenyl]-4,5-dihydro-1H-1,2,4-triazol-3-yl)amino)benzamide

The procedure is as in Example 43, replacing the R(+)-α-amino-butyrolactone in Step D by 3-amino-2-azetidinone.

### EXAMPLE 43

### N-[(3R)-2-oxotetrahydro-3-furanyl]-4-(15-oxo-1-[3-(trifluoromethyl)-phenyl]-4,5-dihydro-1H-1,2,4-triazol-3-yl}amino)benzamide

Step A: Ethyl 4-(([(ethoxycarbonyl)amino]carbothioylyamino)benzoate

To a solution of ethyl 4-aminobenzoate (38.2 g) in CH₃CN (250 ml) there are added ethoxycarbonyl isothiocyanate (30 ml) and diisopropylethylamine (44.3 ml). After stirring for 12 hours at ambient temperature, the precipitate that forms is filtered off and washed with CH₃CN and with Et₂O and then dried in vacuo to yield the title compound.

Step B: Ethyl 4-({5-oxo-1-[3-(trifluoromethyl)phenyl]-4,5-dihydro-1H-1,2,4-triazol-3-yl}amino)benzoate

To a solution of the product obtained in Step A (49.45 g) in dimethylformamide (300 ml) there are added, in succession, 3-(trifluoromethyl)phenylhydrazine (24 ml), EDCI (63.8 g) and diisopropylethylamine (32 ml). The reaction mixture is stirred for 4 hours at ambient temperature. The reaction mixture is poured into 10% aqueous HCl (2 L) and the product is extracted with ethyl acetate (4 times). The organic phase is washed with a 10% solution of HCl (twice) and with water saturated with NaCl. The organic phase is dried over MgSO₄, filtered and evaporated to dryness. The product obtained is dissolved in a 10% solution of trifluoroacetic acid in dioxane and heated at 50° C. overnight. The organic phase is concentrated and the solid obtained is filtered off, washed with ethyl ether (3 times) and then dried in vacuo to yield the title compound.

Step C: 4-({5-Oxo-1-[3-(trifluoromethyl)phenyl]-4,5-dihydro-1H-1,2,4-triazol-3-yl}amino)benzoic Acid

To a solution of the product obtained in Step B (33 g) in a mixture of methanol/tetrahydrofuran (500 ml/500 ml) there is added a solution of lithium hydroxide (24.9 g) in water (100 ml). The reaction mixture is stirred at 50° C. for 12 hours and then concentrated, and the aqueous phase is acidified with concentrated HCl. The precipitate that forms is filtered off and then washed with water and dried in vacuo to yield the title compound.

Step D: N-[(3R)-2-oxotetrahydro-3-furanyl]-4-((5-oxo-1-[3-(trifluoromethyl)-phenyl]-4,5-dihydro-1H-1,2,4-triazol-3-yl}amino)benzamide

To a solution of the compound obtained in Step C (2.65 g) in dimethylformamide (20 ml) there are added 1-(3-dimethylaminopropyl)-3-ethylcarbodiimide hydrochloride (EDCI) (2.08 g), azabenzotriazole (198 mg), diisopropylethylamine (1.27 ml) and R(+)-α-aminobutyrolactone (1 g). The reaction mixture is stirred overnight at ambient temperature. The reaction mixture is poured into 10% aqueous HCl (150 ml). The precipitate that forms is filtered off and washed with water, dried in vacuo and purified by normal phase flash chromatography (CH₂Cl₂ 90/EtOH 10). The product obtained is taken up in dimethyl sulphoxide, poured into 10% aqueous HCl (150 ml), filtered off and washed with water and then dried in vacuo to yield the title compound.

Mass spectrum: ESI-MS:MH⁺=448

### EXAMPLE 44

### N-(2-oxotetrahydro-3-furanyl)-4-({5-oxo-1-[3-(trifluoromethyl)phenyl]-4,5-dihydro-1H-1,2,4-triazol-3-yl}amino)benzamide

The procedure is as in Example 43, replacing the R(+)-α-aminobutyrolactone in Step D by α-aminobutyrolactone.

Mass spectrum: ESI-MS:MH⁺=448

### EXAMPLE 45

### N-[(3S)-2-oxotetrahydro-3-furanyl]-4-({5-oxo-1-[3-(trifluoromethyl)-phenyl]-4,5-dihydro-1H-1,2,4-triazol-3-yl}amino)benzamide

The procedure is as in Example 43, replacing the R(+)-α-aminobutyrolactone in Step D by S(−)-α-aminobutyrolactone.

Mass spectrum: ESI-MS:MH⁺=448

### EXAMPLE 46

### N-[(3S)-2-oxotetrahydro-3-thienyl]-4-({5-oxo-1-[3-(trifluoromethyl)-phenyl]-4,5-dihydro-1H-1,2,4-triazol-3-yl}amino)benzamide

The procedure is as in Example 43, replacing the R(+)-α-aminobutyrolactone in Step D by (3S)-3-aminodihydro-2(3H)-thiophenone.

Mass spectrum: ESI-MS:MH⁺=464

### EXAMPLE 47

### N-(2-oxo-3-pyrrolidinyl)-4-({5-oxo-1-[3-(trifluoromethyl)phenyl]-4,5-dihydro-1H-1,2,4-triazol-3-yl}amino)benzamide

The procedure is as in Example 43, replacing the R(+)-α-aminobutyrolactone in Step D by 3-amino-2-pyrrolidinone.

### EXAMPLE 48

### N-[(4R)-3-oxoisoxazolidinyl]-4-((5-oxo-1-[3-(trifluoromethyl)phenyl]-4,5-dihydro-1H-1,2,4-triazol-3-yl}amino)benzamide

The procedure is as in Example 43, replacing the R(+)-α-aminobutyrolactone in Step D by (R)-4-amino-3-isoxazolidinone.

### EXAMPLE 49

### N-(2-oxo-3-piperidyl)-4-({5-oxo-1-[3-(trifluoromethyl)phenyl]-4,5-dihydro-1H-1,2,4-triazol-3-yl}amino)benzamide

The procedure is as in Example 43, replacing the R(+)-α-aminobutyrolactone in Step D by 3-amino-2-piperidinone.

### EXAMPLE 50

### N-(2-oxo-3-azepanyl)-4-({5-oxo-1-[3-(trifluoromethyl)phenyl]-4,5-dihydro-1H-1,2,4-triazol-3-yl}amino)benzamide

The procedure is as in Example 43, replacing the R(+)-o-aminobutyrolactone in Step D by 3-amino-2-azepanone.

### EXAMPLE 51

### N-[(1R,2S)-2-hydroxy-2,3-dihydro-1H-inden-1-yl]-4-({5-oxo-1-[3-(trifluoromethyl)phenyl]-4,5-dihydro-1H-1,2,4-triazol-3-yl}amino)-benzamide

The procedure is as in Example 43, replacing the R(+)-α-aminobutyrolactone in Step D by (1R,2S)-1-amino-2,3-dihydro-1H-inden-2-ol.

Mass spectrum: ESI-MS:M-H=494

### EXAMPLE 52

### N-[(1S,2R)-2-hydroxy-2,3-dihydro-1H-inden-1-yl]-4-({5-oxo-1-[3-(trifluoromethyl)phenyl]-4,5-dihydro-1H-1,2,4-triazol-3-yl}amino)-benzamide

The procedure is as in Example 43, replacing the R(+)-α-aminobutyrolactone in Step D by (1S,2R)-1-amino-2,3-dihydro-1H-inden-2-ol.

Mass spectrum: ESI-MS:M-H=494

### EXAMPLE 53

### N-(3-hydroxy-2-pyridyl)-4-((5-oxo-1-[3-(trifluoromethyl)phenyl]-4,5-dihydro-1H-1,2,4-triazol-3-yl}amino)benzamide

The procedure is as in Example 43, replacing the R(+)-o-aminobutyrolactone in Step D by 2-amino-3-pyridinol.

Mass spectrum: ESI-MS:MH⁺=457

### EXAMPLE 54

### N-(5-chloro-2-hydroxyphenyl)-4-(15-oxo-1-[3-(trifluoromethyl)phenyl]-4,5-dihydro-1H-1,2,4-triazol-3-yl}amino)benzamide

The procedure is as in Example 43, replacing the R(+)-α-aminobutyrolactone in Step D by 2-amino-4-chlorophenol.

Mass spectrum: ESI-MS:MH⁺=490

### EXAMPLE 55

### 4-{Methyl[1-(3-methylphenyl)-5-oxo-4,5-dihydro-1H-1,2,4-triazol-3-yl]-amino}-N-[(3R)-2-oxotetrahydro-3-furanyl]benzamide

The procedure is as in Steps A and B of Example 43, the product obtained in Step B being subjected to methylation under conventional conditions to obtain ethyl 4-(methyl{5-oxo-1-[3-(trifluoromethyl)phenyl]-4,5-dihydro-1H-1,2,4-triazol-3-yl} amino)benzoate; the procedure is then as in Steps C and D of Example 43.

### EXAMPLE 56

### N-[(1R,2S)-2-hydroxy-2,3-dihydro-1H-inden-1-yl]-4-{[1-(3-nitrophenyl)-5-oxo-4,5-dihydro-1H-1,2,4-triazol-3-yl]amino}benzamide

The procedure is as in Example 43, replacing the 3-(trifluoromethyl)phenylhydrazine in Step B by 1-(3-nitrophenyl)hydrazine, and replacing the R(+)-α-aminobutyrolactone in Step D by (I R,2S)-1-amino-2,3-dihydro-1H-inden-2-ol.

### EXAMPLE 57

### 4-({1-[4-(Aminosulphonyl)phenyl]-5-oxo-4,5-dihydro-1H-1,2,4-triazol-3-yl}amino)-N-[(1R,2S)-2-hydroxy-2,3-dihydro-1H-inden-1-yl]benzamide

The procedure is as in Example 43, replacing the 3-(trifluoromethyl)phenylhydrazine in Step B by 4-hydrazinobenzenesulphonamide, and replacing the R(+)-α-aminobutyrolactone in Step D by (1R,2S)-1-amino-2,3-dihydro-1H-inden-2-ol.

Mass spectrum: ESI-MS:M-H=505

### EXAMPLE 58

### 4-{3-[4-({[(1R,2S)-2-Hydroxy-2,3-dihydro-1H-inden-1-yl]amino}-carbonyl)anilino]-5-oxo-4,5-dihydro-1H-1,2,4-triazol-1-yl}benzene-sulphonic Acid

The procedure is as in Example 43, replacing the 3-(trifluoromethyl)phenylhydrazine in Step B by 4-hydrazinobenzenesulphonic acid, and replacing the R(+)-α-aminobutyrolactone in Step D by (1R,2S)-1-amino-2,3-dihydro-1H-inden-2-ol.

### EXAMPLE 59

### N-[(1R,2S)-2-hydroxy-2,3-dihydro-1H-inden-1-yl]-4-({5-oxo-1-[5-(trifluoromethyl)-2-pyridyl]-4,5-dihydro-1H-1,2,4-triazol-3-yl}-amino)benzamide

The procedure is as in Example 43, replacing the 3-(trifluoromethyl)phenylhydrazine in Step B by 2-hydrazino-5-(trifluoromethyl)pyridine, and replacing the R(+)-α-aminobutyrolactone in Step D by (I R,2S)-1-amino-2,3-dihydro-1H-inden-2-ol.

Mass spectrum: ESI-MS:MH⁺=497

### EXAMPLE 60

### N-[(1R,2S)-2-hydroxy-2,3-dihydro-1H-inden-1-yl]4-{[1-(2-naphthyl)-5-oxo-4,5-dihydro-1H-1,2,4-triazol-3-yl]amino}benzamide

The procedure is as in Example 43, replacing the 3-(trifluoromethyl)phenylhydrazine in Step B by 1-(2-naphthyl)hydrazine, and replacing the R(+)-α-aminobutyrolactone in Step D by (1R,2S)-1-amino-2,3-dihydro-1H-inden-2-ol.

### EXAMPLE 61

### N-[(1R,2S)-2-hydroxy-2,3-dihydro-1H-inden-1-yl]-4-{[5-oxo-1-(2-quinolyl)-4,5-dihydro-1H-1,2,4-triazol-3-yl]amino}benzamide

The procedure is as in Example 43, replacing the 3-(trifluoromethyl)phenylhydrazine in Step B by 2-hydrazinoquinoline, and replacing the R(+)-α-aminobutyrolactone in Step D by (1R,2S)-1-amino-2,3-dihydro-1H-inden-2-ol.

### EXAMPLE 62

### 4-{[1-(1,3-Benzothiazol-2-yl)-5-oxo-4,5-dihydro-1H-1,2,4-triazol-3-yl]-amino}-N-[(1R,2S)-2-hydroxy-2,3-dihydro-1H-inden-1-yl]benzamide

The procedure is as in Example 43, replacing the 3-(trifluoromethyl)phenylhydrazine in Step B by 2-hydrazino-1,3-benzothiazole, and replacing the R(+)-α-aminobutyrolactone in Step D by (1R,2S)-1-amino-2,3-dihydro-1H-inden-2-ol.

Mass spectrum: ESI-MS:M-H=483

### EXAMPLE 63

### 5-[4-({([(1S,2R)-2-Hydroxy-2,3-dihydro-1H-inden-1-yl]amino}methyl)-anilino]-2-phenyl-2,4-dihydro-3H-1,2,4-triazol-3-one

The procedure is as in Example 76, replacing the 4-({5-oxo-1-[3-(trifluoromethyl)phenyl]-4,5-dihydro-1H-1,2,4-triazol-3-yl}amino)benzoic acid in Step A (obtained in Step C of Example 43) by 4-[(5-oxo-1-phenyl-4,5-dihydro-1H-1,2,4-triazol-3-yl)amino]benzoic acid (obtained in Step C of Example 4), and replacing the R(+)-α-aminobutyrolactone in Step C by (1S,2R)-1-amino-2,3-dihydro-1H-inden-2-ol.

Mass spectrum: ESI-MS:MH⁺=414

### EXAMPLE 64

### 5-[4-({[(1R,2S)-2-Hydroxy-2,3-dihydro-1H-inden-1-yl]amino)methyl)-anilino]-2-phenyl-2,4-dihydro-3H-1,2,4-triazol-3-one trifluoroacetate

The procedure is as in Example 63, replacing the (1S,2R)-1-amino-2,3-dihydro-1H-inden-2-ol in Step C by (1R,2S)-1-amino-2,3-dihydro-1H-inden-2-ol.

Mass spectrum: ESI-MS:MH⁺=414

### EXAMPLE 65

### (1R*,2S*)-2-[(4-{[1-(3-Methylphenyl)-5-oxo-4,5-dihydro-1H-1,2,4-triazol-3-yl]amino}benzyl)amino] cyclopentanecarboxamide trifluoroacetate

The procedure is as in Example 76, replacing the 4-({5-oxo-1-[3-(trifluoromethyl)phenyl]-4,5-dihydro-1H-1,2,4-triazol-3-yl}amino)benzoic acid in Step A (obtained in Step C of Example 43) by 4-{[1-(3-methylphenyl)-5-oxo-4,5-dihydro-1H-1,2,4-triazol-3-yl]amino}-benzoic acid (obtained in Step C of Example 18), and replacing the R(+)-α-aminobutyrolactone in Step C by (1R*,2S*)-2-aminocyclopentanecarboxamide.

Mass spectrum: ESI-MS:MHW=407

### EXAMPLE 66

### 5-[4-({[(1R*,2R *)-2-Hydroxycyclohexyl]amino}methyl)anilino]-2-(3-methylphenyl)-2,4-dihydro-3H-1,2,4-triazol-3-one trifluoroacetate

The procedure is as in Example 65, replacing the (1R*,2S*)-2-aminocyclopentanecarboxamide in Step C by (1R*,2R*)-2-aminocyclohexanol.

Mass spectrum: ESI-MS:MH⁺=394

### EXAMPLE 67

### 3-[(4-{[1-(3-Methylphenyl)-5-oxo-4,5-dihydro-1H-1,2,4-triazol-3-yl]amino}benzyl)amino]-2-azepanone trifluoroacetate

The procedure is as in Example 65, replacing the (1R*,2S*)-2-aminocyclopentanecarboxamide in Step C by 3-amino-2-azepanone.

Mass spectrum: ESI-MS:MH⁺=407

### EXAMPLE 68

### 5-4-[(5-Acetyl-2-hydroxyanilino)methyl]anilino}-2-(3-methylphenyl)-2,4-dihydro-3H-1,2,4-triazol-3-one

The procedure is as in Example 65, replacing the (1R*,2S*)-2-aminocyclopentanecarboxamide in Step C by 1-(3-amino-4-hydroxyphenyl)ethanone.

Mass spectrum: ESI-MS:MH⁺=430

### EXAMPLE 69

### 5-{4-[(5-Chloro-2-hydroxyanilino)methyl]anilino}-2-(3-methylphenyl)-2,4-dihydro-3H-1,2,4-triazol-3-one

The procedure is as in Example 65, replacing the (1R*,2S*)-2-aminocyclopentanecarboxamide in Step C by 2-amino-4-chlorophenol.

Mass spectrum: ESI-MS:MH⁺=422

### EXAMPLE 70

### 5-[4-({[(1R,2S)-2-Hydroxy-2,3-dihydro-1H-inden-1-yl]amino}methyl)-anilino]-2-(3-methylphenyl)-2,4-dihydro-3H-1,2,4-triazol-3-one trifluoroacetate

The procedure is as in Example 65, replacing the (1R*,2S*)-2-aminocyclopentanecarboxamide in Step C by (1R,2S)-1-amino-2,3-dihydro-1H-inden-2-ol.

Mass spectrum: ESI-MS:MH⁺=428

### EXAMPLE 71

### 3-[(4-{[1-(3-Chlorophenyl)-5-oxo-4,5-dihydro-1H-1,2,4-triazol-3-yl]-amino}benzyl)amino]-2-azepanone trifluoroacetate

The procedure is as in Example 76, replacing the 4-({5-oxo-1-[3-(trifluoromethyl)phenyl]-4,5-dihydro-1H-1,2,4-triazol-3-yl}amino)benzoic acid in Step A (obtained in Step C of Example 43) by 4-{[1-(3-chlorophenyl)-5-oxo4,5-dihydro-1H-1,2,4-triazol-3-yl]amino}-benzoic acid (obtained in Step C of Example 29), and replacing the R(+)-α-aminobutyrolactone in Step C by 3-amino-2-azepanone.

Mass spectrum: ESI-MS:MH⁺=427

### EXAMPLE 72

### 2-(3-Chlorophenyl)-5-[4-({[(1S,2R)-2-hydroxy-2,3-dihydro-1H-inden-1-yl]amino}methyl)anilino]-2,4-dihydro-3H-1,2,4-triazol-3-one trifluoroacetate

The procedure is as in Example 71, replacing the 3-amino-2-azepanone in Step C by (1S,2R)-1-amino-2,3-dihydro-1H-inden-2-ol.

Mass spectrum: ESI-MS:MH⁺=448

### EXAMPLE 73

### 2-(3-Chlorophenyl)-5-[4-({[(1R,2S)-2-hydroxy-2,3-dihydro-1H-inden-1-yl]amino}methyl)anilino]-2,4-dihydro-3H-1,2,4-triazol-3-one trifluoroacetate

The procedure is as in Example 71, replacing the 3-amino-2-azepanone in Step C by (1R,2S)-1-amino-2,3-dihydro-1H-inden-2-ol.

Mass spectrum: ESI-MS:MH⁺=448

### EXAMPLE 74

### 2-(4-Chlorophenyl)-5-{4-[(2-hydroxyanilino)methyl]anilino}-2,4-dihydro-3H-1,2,4-triazol-3-one trifluoroacetate

The procedure is as in Example 76, replacing the 4-({5-oxo-1-[3-(trifluoromethyl)phenyl]-4,5-dihydro-1H-1,2,4-triazol-3-yl}amino)benzoic acid in Step A (obtained in Step C of Example 43) by 4-{[1-(4-chlorophenyl)-5-oxo-4,5-dihydro-1H-1,2,4-triazol-3-yl]amino}-benzoic acid (obtained in Step C of Example 34), and replacing the R(+)-α-aminobutyrolactone in Step C by 2-aminophenol.

Mass spectrum: ESI-MS:MH⁺=408

### EXAMPLE 75

### 5-[4-({Benzyl-[(1S*,2S*)-2-hydroxycyclohexyl]amino}methyl)anilino]-2-(4-chlorophenyl)-2,4-dihydro-3H-1,2,4-triazol-3-one trifluoroacetate

The procedure is as in Example 74, replacing the 2-aminophenol in Step C by (1S*,2S*)-2-(benzylamino)cyclohexanol.

Mass spectrum: ESI-MS:MH⁺=504

### EXAMPLE 76

### 5-[4-({[(3R)-2-Oxotetrahydro-3-furanyl]amino}methyl)anilino]-2-[3-(trifluoromethyl)phenyl]-2,4-dihydro-3H-1,2,4-triazol-3-one hydrochloride

Step A: N-Methoxy-N-methyl-4-({5-oxo-1-[3-(trifluoromethyl)phenyl]-4,5-dihydro-1H-1, 2, 4-triazol-3-yl}amino)benzamide

To a solution of the product obtained in Step C of Example 43 (12.2 g) in dimethylformamide (100 ml) there are added EDCI (9.63 g), azabenzotriazole (4.57 g), diisopropylethylamine (8.7 ml) and N,O-dimethylhydroxylamine hydrochloride (4.9 g). The reaction mixture is stirred overnight at room temperature and is then poured into 10% aqueous HCl (150 ml). The precipitate that forms is filtered off, washed with water and then dried in vacuo to yield the title compound.

Step B: 4-((5-Oxo-1-[3-(trifluoromethyl)phenyl]-4,5-dihydro-1H-1, 2, 4-triazol-3-yl) amino)benzaldehyde

To a solution of lithium aluminium hydride (IM) in tetrahydrofuran (50 ml) there is added, dropwise, a solution of the compound obtained in Step A in tetrahydrofuran (100 ml) under an inert atmosphere and at −40° C. After stirring for one hour at −40° C., the reaction mixture is brought to 0° C. until the starting material has completely disappeared and then cooled to −10° C., and the excess of reducing agent is destroyed by slowly adding water. The expected product is extracted with ethyl acetate. The organic phase is washed with water and then washed with saturated aqueous sodium chloride solution; it is dried over magnesium sulphate, filtered and evaporated in vacuo to yield the title compound.

Step C: 5-[4-({[(3R)-2-Oxotetrahydro-3-furanyl]amino}methyl)anilino]-2-[3-(trifluoromethyl)phenyl]-2,4-dihydro-3H-1, 2,4-triazol-3-one hydrochloride

To a solution of the product obtained in Step B (2 g) in dichloromethane (20 ml) there are added R(+)-α-aminobutyrolactone (870 mg) and sodium borohydride (2.43 g) at ambient temperature, and the reaction mixture is then stirred at ambient temperature until the starting material has completely disappeared. The reaction mixture is then concentrated and taken up in ethyl acetate, and the excess of reducing agent is destroyed by adding saturated aqueous sodium bicarbonate solution. The organic phase is washed with water and then with saturated aqueous sodium chloride solution; it is then dried over magnesium sulphate, filtered and evaporated in vacuo. The residue obtained is taken up in a 4M solution of HCl in dioxane, evaporated to dryness and dried in vacuo; it is then triturated in ethyl acetate, filtered and dried in vacuo to yield the title compound.

Mass spectrum: ESI-MS:MH⁺=434

### EXAMPLE 77

### 5-[4-({[(4R)-3-Oxoisoxazolidinyl]amino}methyl)anilinol-2-[3-(trifluoromethyl)phenyl]-2,4-dihydro-3H-1,2,4-triazol-3-one hydrochloride

The procedure is as in Example 76, replacing the R(+)-α-aminobutyrolactone in Step C by (R)-4-amino-3-isoxazolidinone.

Mass spectrum: ESI-MS:MH⁺=435

### EXAMPLE 78

### 3-{([4-({5-Oxo-1-[3-(trifluoromethyl)phenyl]-4,5-dihydro-1H-1,2,4-triazol-3-yl}amino)benzyl]amino}-2-azepanone

The procedure is as in Example 76, replacing the R(+)-α-aminobutyrolactone in Step C by 3-amino-2-azepanone.

Mass spectrum: ESI-MS:MH⁺=461

### EXAMPLE 79

### (1R*,2S*)-2-{[4-({5-Oxo-1-3-(trifluoromethyl)phenyl]-4,5-dihydro-1H-1,2,4-triazol-3-yl}amino)benzyl]amino}cyclopentanecarboxamide trifluoroacetate

The procedure is as in Example 76, replacing the R(+)-α-aminobutyrolactone in Step C by (1R*,2S*)-2-aminocyclopentanecarboxamide.

Mass spectrum: ESI-MS:MH⁺=461

### EXAMPLE 80

### 5-[4-({[(1R *,2R *)-2-Hydroxycyclohexyl]amino}methyl)anilino]-2-[3-(trifluoromethyl)phenyl]-2,4-dihydro-3H-1,2,4-triazol-3-one trifluoroacetate

The procedure is as in Example 76, replacing the R(+)-α-aminobutyrolactone in Step C by (1R*,2R*)-2-aminocyclohexanol.

Mass spectrum: ESI-MS:MH⁺=448

### EXAMPLE 81

### (1R*,2S*)-2-{[4-({5-Oxo-1-[3-(trifluoromethyl)phenyl]-4,5-dihydro-1H-1,2,4-triazol-3-yl}amino)benzyl]amino}cyclohexanecarboxamide trifluoroacetate

The procedure is as in Example 76, replacing the R(+)-α-aminobutyrolactone in Step C by (1 R *,2S*)-2-aminocyclohexanecarboxamide.

Mass spectrum: ESI-MS:MHW=475

### EXAMPLE 82

### 5-(4-{[(3-Hydroxy-2-pyridyl)amino]methyl}anilino)-2-[3-(trifluoromethyl)phenyl]-2,4-dihydro-3H-1,2,4-triazol-3-one trifluoroacetate

The procedure is as in Example 76, replacing the R(+)-α-aminobutyrolactone in Step C by 2-amino-3-pyridinol.

Mass spectrum: ESI-MS:MH⁺=443

### EXAMPLE 83

### 5-[4-({[(1R,2S)-2-Hydroxy-2,3-dihydro-1H-inden-1-yl]amino)methyl)-anilino]-2-[3-(trifluoromethyl)phenyl]-2,4-dihydro-3H-1,2,4-triazol-3-one trifluoroacetate

The procedure is as in Example 76, replacing the R(+)-α-aminobutyrolactone in Step C by (1R,2S)-1-amino-2,3-dihydro-1H-inden-2-ol.

Mass spectrum: ESI-MS:MH⁺=482

### EXAMPLE 84

### 5-[4-({Benzyl-[(1S*,2S*)-2-hydroxycyclohexyl]amino}methyl)anilinol-2-13-(trifluoromethyl)phenyl]-2,4-dihydro-3H-1,2,4-triazol-3-one trifluoroacetate

The procedure is as in Example 76, replacing the R(+)-α-aminobutyrolactone in Step C by (1S*,2S*)-2-(benzylamino)cyclohexanol.

Mass spectrum: ESI-MS:MH⁺=538

### Pharmacological Study

### EXAMPLE A

### Measurement of the Effect on Food Intake in Wistar Rats Fasted for 24 Hours

The products of the invention were tested in vivo in Wistar rats subjected to dietary restriction for 24 hours, in order to assess the influence thereof on food intake. The animals used are male Wistar rats (275-300 g).

The rats are placed in individual cages fitted with a grating floor and with free access to food and liquid. The animals are kept in the animal quarters under controlled conditions of temperature, humidity and light for a period of 6 days before carrying out the tests. The experiments are summarised as follows:

D-1 at time T=0: Fasting of the rats started in the morning

D 0 at time T=0: Animals treated with the compound under test, the control group being given the carrier (10% DMSO+10% Solutol HS 15)

D 0 at time T=1 hour after treatment: Reintroduction of food, the dishes being filled with food and weighed before being replaced in each cage

D 0 at time T=2 hours after treatment: First measurement of food intake

D 0 at time T=3, 4, 5 and 7 hours after treatment: Cumulative measurement of food intake.

The test compounds are dissolved, immediately before use, in 10% DMSO+10% Solutol HS 15, as a function of their solubility, and are administered intraperitoneally (IP) at a dose of 5 or 7.5 mg/kg and in a volume of 2.0 ml/kg.

Results:

The compounds of the invention show very good percentage inhibition of food intake: the percentage inhibition of food intake in the treated group with respect to the food intake control consisting of the carrier is calculated for each point in time (2, 3, 4, 5 or 7 hours after treatment) and analysed by means of a single-factor (factor: treatment) ANOVA test.

### EXAMPLE B

### Measurement of the In Vitro Affinity for NPY Receptors

The capacity of the compounds of the invention to bind to NPY receptors was measured in various cell lines, each expressing one of the receptor sub-types studied. Competition binding experiments were carried out using the peptide [¹²⁵I]-PYY as radioligand at concentrations ranging from 15 to 65 pM. The non-specific fraction is measured in the presence of a concentration of 1 μM NPY. The cells are incubated for a period ranging from 1 to 2 hours depending upon the lines, and the radioactivity is collected after filtration over a GF/C filter treated with 0.1% PEI, before being measured.

Results:

The results are expressed as IC₅₀. The compounds of the invention appear to be capable of significantly displacing the reference ligand: the IC₅₀ values vary from a few nanomoles to some hundreds of nanomoles.

### EXAMPLE C

### Acute Toxicity Study

Acute toxicity was evaluated after oral administration of increasing doses of the test compound to groups each comprising 8 mice (26±6 grams). The animals were observed at regular intervals over the course of the first day and daily for the two weeks following treatment.

The toxicity of the compounds of the invention appears to be very low.

### EXAMPLE D

### Pharmaceutical Composition

Formulation for the preparation of 1000 tablets each comprising a dose of 10 mg of N-[(3R)-2-oxotetrahydro-3-furanyl]-4-({5-oxo-1-[3-(trifluoromethyl)phenyl]-4,5-dihydro-1H-1,2,4-triazol-3-yl} amino)benzamide (Example 43)

| | |
|---|---|
| Compound of Example 43 | 10 g |
| Hydroxypropyl cellulose | 2 g |
| Wheat starch | 10 g |
| Lactose | 100 g |
| Magnesium stearate | 3 g |
| Talc | 3 g |

1. Compounds of formula (I): 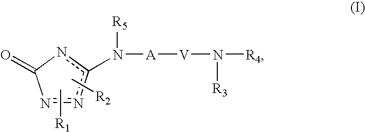
wherein:
R₁ and R₂ each independently of the other represents a hydrogen atom or an unsubstituted or substituted alkyl group, an unsubstituted or substituted alkenyl group, an unsubstituted or substituted alkynyl group, an unsubstituted or substituted aryl group, an unsubstituted or substituted heteroaryl group, an unsubstituted or substituted cycloalkyl group, or an unsubstituted or substituted heterocycloalkyl group, it being understood that at least one of groups R₁ and R₂ is other than a hydrogen atom,
R₃ represents a hydrogen atom or an unsubstituted or substituted alkyl group, an unsubstituted or substituted alkenyl group, an unsubstituted or substituted alkynyl group, an unsubstituted or substituted aryl group, an unsubstituted or substituted heteroaryl group, an unsubstituted or substituted cycloalkyl group, or an unsubstituted or substituted heterocycloalkyl group,
R₄ represents a group of formula (II): 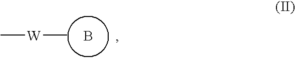
wherein W represents a bond or an alkylene chain containing from 1 to 6 carbon atoms and B represents a mono- or poly-cyclic, aromatic or non-aromatic, group containing from 3 to 10 ring atoms, which may include from 1 to 3 hetero atoms selected from oxygen, sulphur and nitrogen, and containing at least one oxo, —COR (wherein R represents a hydrogen atom or an alkyl, alkoxy, amino, alkylamino or dialkylamino group) or hydroxy substituent, and which may contain one or more unsaturations and/or one or more substituents (in addition to the oxo, COR or hydroxy group defined above) selected from alkyl, alkoxy, aryl, arylalkyl and halogen atoms,
R₅ represents a hydrogen atom or an alkyl group,
A represents a group selected from -A₂-, -A₁-A₂-, -A₂-A₁- and -A₁-A₂-A₁-, wherein A₁ is an alkylene, alkenylene or alkynylene group and A₂ represents an unsubstituted or substituted phenylene group, an unsubstituted or substituted naphthylene group, an unsubstituted or substituted cycloalkylene group, an unsubstituted or substituted heteroarylene group, or an unsubstituted or substituted heterocycloalkylene group,
V represents a bond or a group —CH₂—, —CO—, —CS—, —CH₂—NH— or —CH═N—, or V and R₃, together with the groups -A- and —N—R₄ carrying them, form a group -A-CH═N—R₄,
it being understood that,
the term “alkyl” denotes a linear or branched group having from 1 to 6 carbon atoms,
the term “alkylene” denotes a linear or branched bivalent radical containing from 1 to 6 carbon atoms,
the term “alkenyl” denotes a linear or branched group containing from 2 to 6 carbon atoms and from 1 to 3 double bonds,
the term “alkenylene” denotes a linear or branched bivalent radical containing from 2 to 6 carbon atoms and from 1 to 3 double bonds,
the term “alkynyl” denotes a linear or branched group containing from 2 to 6 carbon atoms and from 1 to 3 triple bonds,
the term “alkynylene” denotes a linear or branched bivalent radical containing from 2 to 6 carbon atoms and from 1 to 3 triple bonds,
the term “aryl” denotes a phenyl, naphthyl, biphenyl, dihydronaphthyl or tetrahydronaphthyl group,
the term “heteroaryl” denotes an unsaturated or partially unsaturated mono- or bi-cyclic group having from 5 to 11 ring members, containing from 1 to 4 hetero atoms selected from nitrogen, oxygen and sulphur,
the terms “phenylene” and “naphthylene” denote bivalent phenyl and naphthyl radicals, respectively,
the term “heteroarylene” denotes a bivalent heteroaryl radical, heteroaryl being as defined hereinbefore,
the term “heterocycloalkyl” denotes a saturated mono- or bi-cyclic group having from 4 to 11 ring members, containing from 1 to 4 hetero atoms selected from nitrogen, oxygen and sulphur,
the term “heterocycloalkylene” denotes a saturated mono- or bi-cyclic bivalent radical having from 4 to 11 ring members, containing from 1 to 4 hetero atoms selected from nitrogen, oxygen and sulphur,
the term “cycloalkyl” denotes a saturated cyclic group containing from 3 to 8 carbon atoms,
the term “cycloalkylene” denotes a saturated bivalent cyclic group containing from 3 to 8 carbon atoms,
the expression “substituted” applied to the terms “aryl” or “heteroaryl” means that those groups are substituted on their cyclic moiety by from 1 to 5 identical or different substituents selected from linear or branched (C₁-C₆)alkyl, linear or branched (C₁-C₆)alkoxy, halogen, hydroxy, linear or branched (C₁-C₆)-perhaloalkyl, nitro, amino (unsubstituted or substituted by one or two groups selected from linear or branched (C₁-C₆)alkyl, aryl and heteroaryl), linear or branched (C₁-C₆)acyl, aminocarbonyl (optionally substituted on the nitrogen atom by one or two linear or branched (C₁-C₆)alkyl groups), linear or branched (C₁-C₆)acylamino, linear or branched (C₁-C₆)alkoxy-carbonyl, formyl, carboxy, sulpho, sulphino, sulphamoyl, nitrile, linear or branched (C₁-C₆)-aminoalkyl (optionally substituted on the nitrogen atom by one or two linear or branched (C₁-C₆)alkyl groups), linear or branched (C₁-C₆)-thioalkyl (optionally substituted on the sulphur atom by a linear or branched (C₁-C₆)alkyl group) and hydroxyalkyl (optionally substituted on the oxygen atom by a linear or branched (C₁-C₆)alkyl group), the expression “substituted” applied to the terms “alkyl”, “alkenyl” or “alkynyl” means that those groups may be substituted by one or more groups selected from hydroxy, unsubstituted or substituted cycloalkyl, unsubstituted or substituted aryl, unsubstituted or substituted heteroaryl, unsubstituted or substituted heterocycloalkyl and halogen atoms,
the expression “substituted” applied to the terms “phenylene”, “naphthylene” or “heteroarylene” means that those groups are substituted by from one to three identical or different groups selected from linear or branched (C₁-C₆)alkyl, linear or branched (C₁-C₆)alkoxy, halogen, hydroxy, linear or branched (C₁-C₆)-perhaloalkyl, nitro, amino (unsubstituted or substituted by one or two groups selected from linear or branched (C₁-C₆)alkyl, aryl and heteroaryl), linear or branched (C₁-C₆)acyl, formyl, carboxy, linear or branched (C₁-C₆)alkoxy-carbonyl, aminocarbonyl (optionally substituted on the nitrogen atom by one or two linear or branched (C₁-C₆)alkyl groups), linear or branched (C₁-C₆)acylamino and nitrile,
their enantiomers, diastereoisomers, and addition salts thereof with a pharmaceutically acceptable acid or base.

2. Compounds of formula (I) according to claim 1, wherein A represents a phenylene group, their enantiomers, diastereoisomers, and addition salts thereof with a pharmaceutically acceptable acid or base.

3. Compounds of formula (I) according to claims 1 and 2, wherein R₁ represents a hydrogen atom and R₂ represents an aryl group, their enantiomers, diastereoisomers, and addition salts thereof with a pharmaceutically acceptable acid or base.

4. Compounds of formula (I) according to any one of claims 1 to 3, wherein R₄ represents a group of formula (II′): 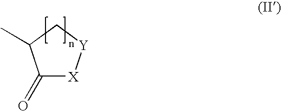
wherein n is 0, 1, 2 or 3, and X represents an oxygen or sulphur atom and in this case Y represents a group CH₂, or X represents a group NH and in this case Y represents a group CH₂ or an oxygen atom, their enantiomers, diastereoisomers, and addition salts thereof with a pharmaceutically acceptable acid or base.

5. Compounds of formula (I) according to any one of claims 1 to 3, wherein R₄ represents a group of formula (II″): 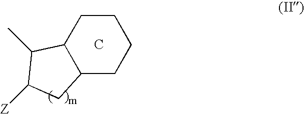
wherein m is 0, 1 or 2, and Z represents a hydroxy or amino group and C represents an optionally substituted, aromatic 6-membered ring which may contain from 1 to 3 nitrogen atoms.

6. Compounds of formula (I) according to any one of claims 1 to 5, wherein V represents a group CO or CH₂.

7. Compounds of formula (I) according to any one of claims 1 to 4 and 6, that are:
N-[(3R)-2-oxotetrahydro-3-furanyl]-4-({5-oxo-1-[3-(trifluoromethyl)phenyl]-4,5-dihydro-1H-1,2,4-triazol-3-yl}amino)benzamide
4-([1-(3-methylphenyl)-5-oxo-4,5-dihydro-1H-1,2,4-triazol-3-yl]amino}-N-[(3R)-2-oxotetrahydro-3-furanyl]benzamide
4-{[1-(3-methylphenyl)-5-oxo-4,5-dihydro-1H-1,2,4-triazol-3-yl]amino}-N-[(3R)-2-oxotetrahydro-3-thienyl]benzamide
4-{[1-(3-chlorophenyl)-5-oxo-4,5-dihydro-1H-1,2,4-triazol-3-yl]amino}-N-[(3R)-2-oxotetrahydro-3-thienyl]benzamide trifluoroacetate
4-{[1-(3-chlorophenyl)-5-oxo-4,5-dihydro-1H-1,2,4-triazol-3-yl]amino}-N-[(3R)-2-oxotetrahydro-3-furanyl]benzamide
4-{[1-(4-chlorophenyl)-5-oxo-4,5-dihydro-1H-1,2,4-triazol-3-yl]amino}-N-[(3R)-2-oxotetrahydro-3-furanyl]benzamide
3-[(4-{[1-(3-methylphenyl)-S-oxo-4,5-dihydro-1H-1,2,4-triazol-3-yl]amino}benzyl)amino]-2-azepanone trifluoroacetate
and addition salts thereof with a pharmaceutically acceptable acid.

8. Compounds of formula (I) according to any one of claims 1 to 3, 5 and 6, that are:
N-[(1R,2S)-2-hydroxy-2,3-dihydro-1H-inden-1-yl]-4-[(5-oxo-1-phenyl-4,5-dihydro-1H-1,2,4-triazol-3-yl)amino]benzamide
N-[(1R,2S)-2-hydroxy-2,3-dihydro-1H-inden-1-yl]-4-{[1-(3-methylphenyl)-5-oxo-4,5-dihydro-1H-1,2,4-triazol-3-yl]amino}benzamide
4-{[1-(3-chlorophenyl)-5-oxo-4,5-dihydro-1H-1,2,4-triazol-3-yl]amino}-N-[(1R,2S)-1-hydroxy-2,3-dihydro-1H-inden-2-yl]benzamide
2-(3-chlorophenyl)-5-[4-({[(1S,2R)-2-hydroxy-2,3-dihydro-1H-inden-1-yl]amino}-methyl)anilino]-2,4-dihydro-3H-1,2,4-triazol-3-one trifluoroacetate
2-(3-chlorophenyl)-5-[4-({[(1R,2S)-2-hydroxy-2,3-dihydro-1H-inden-1-yl]amino}-methyl)anilino]-2,4-dihydro-3H-1,2,4-triazol-3-one trifluoroacetate
and addition salts thereof with a pharmaceutically acceptable acid.

9. Process for the preparation of compounds of formula (I) according to claim 1, characterised in that there is used as starting material the compound of formula (III): 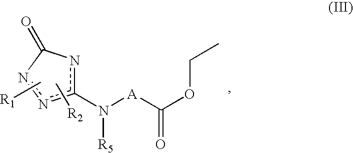
wherein R₁, R₂, R₅ and A are as defined hereinbefore,
which is hydrolysed in a basic medium to yield the compound of formula (IV): 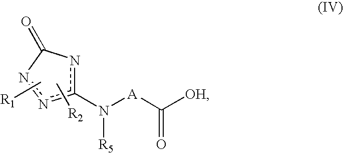
wherein R₁, R₂, R₅ and A are as defined hereinbefore,
with which there is condensed, in the presence of a coupling agent, an amine of formula NHR′₃R₄ (wherein R₄ is as defined hereinbefore and R′₃ may have any of the meanings of R₃ but cannot form a further bond together with V) to yield the compound of formula (I/a), a particular case of the compounds of formula (I): 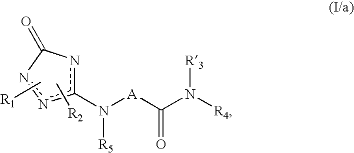
wherein R₁, R₂, R′₃, R₄, R₅ and A are as defined hereinbefore,
which is subjected to a thionating agent such as Lawesson's reagent to yield the compound of formula (IIb), a particular case of the compounds of formula (I): 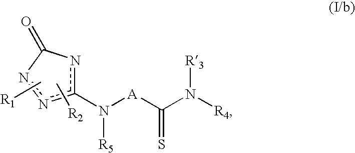
wherein R₁, R₂, R′₃, R₄, R₅ and A are as defined hereinbefore,
or which is subjected to a reducing agent to yield the compound of formula (I/c), a particular case of the compounds of formula (I): 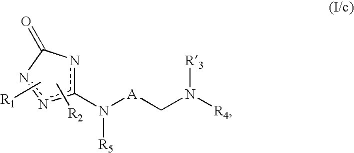
wherein R₁, R₂, R′₃, R₄, R₅ and A are as defined hereinbefore,
or with which compound of formula (IV) there is condensed, in the presence of a coupling agent, N,O-dimethylhydroxylamine, and which is then reduced in the presence of a reducing agent to yield the compound of formula (V): 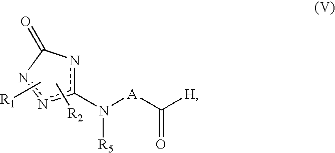
wherein R₁, R₂, R₅ and A are as defined hereinbefore,
with which there is condensed a compound of formula R₄NH₂, wherein R₄ is as defined hereinbefore, to yield the compound of formula (I/d), a particular case of the compounds of formula (1): 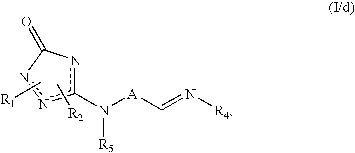
wherein R₁, R₂, R₄, R₅ and A are as defined hereinbefore,
which may be reduced to obtain the compound of formula (I/c′), a particular case of the compounds of formula (I/c): 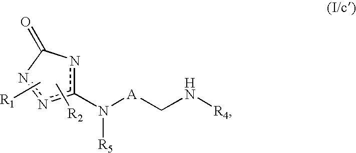
wherein R₁, R₂, R₄, R₅ and A are as defined hereinbefore,
or with which there is condensed a hydrazine of formula H₂N—NR′₃R₄ wherein R′₃ and R₄ are as defined hereinbefore,
under non-reductive conditions to yield the compound of formula (I/e), a particular case of the compounds of formula (1): 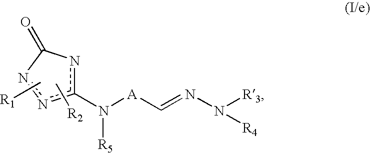
wherein R₁, R₂, R′₃, R₄, R₅ and A are as defined hereinbefore,
or in the presence of a reducing agent to yield the compound of formula (I/f), a particular case of the compounds of formula (I): 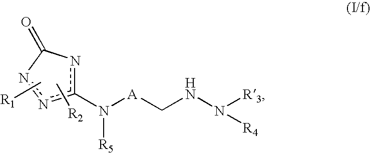
wherein R₁, R₂, R′₃, R₄, R₅ and A are as defined hereinbefore,
the totality of compounds (I/a) to (I/f) constituting the compound of formula (I), which may be purified, if desired, according to a conventional purification technique, which may be separated, where applicable, into its isomers (enantiomers and/or diastereoisomers) according to a conventional separation technique, and which is converted, where appropriate, into addition salts thereof with a pharmaceutically acceptable acid or base.

10. Pharmaceutical compositions comprising as active ingredient at least one compound according to any one of claims 1 to 8, alone or in combination with one or more inert, non-toxic, pharmaceutically acceptable excipients or carriers.

11. Pharmaceutical compositions according to claim 10 comprising at least one active ingredient according to any one of claims 1 to 8 for use as a ligand of receptors of neuropeptide Y in the treatment of pathologies associated with eating behaviour disorders or energy balance disorders, such as diabetes, obesity, bulimia and anorexia nervosa, or in the treatment of arterial hypertension, anxiety, depression, epilepsy, sexual dysfunctions and sleep disorders.
